(19) United States
(12) Patent Application Publication
(10) Pub. No.: US 2021/0315734 A1
(43) Pub. Date: Oct. 14, 2021

(54) Method for Solid Water Particles Treatment of Eye, Ear, and Nose Ailments

(71) Applicant: Shui Yin Lo, Arcadia, CA (US)

(72) Inventor: Shui Yin Lo, Arcadia, CA (US)

(21) Appl. No.: 17/222,852

(22) Filed: Apr. 5, 2021

Related U.S. Application Data
(60) Provisional application No. 63/007,554, filed on Apr. 9, 2020.

(51) Int. Cl. A61F 9/00; A61B 5/01; A61B 5/00; A61M 31/00; A61M 11/00

(52) U.S. Cl. CPC A61F 9/0008; A61B 5/015; A61B 5/4848; A61M 2210/0618; A61M 11/00; A61B 5/4854; A61M 2210/0662; A61M 31/00

(57) ABSTRACT

A method for solid water particle treatment of eye, ear, and nose ailments detects and treats ailments in the eyes, ears, and nose through a procedure of capturing infrared images of the Meridian pathway near the eyes, ears, or nose, and then administering a solid water particle solution to help repair the Meridian pathway. The efficacy of the solid water particle solution is determined by capturing an initial image and a subsequent image of the Meridian pathway near the eyes, ears, or nose. The skin temperature of both images is visually indicated in a thermal coloring or white-hot spot. If the temperatures detected in the subsequent infrared images decrease, the Meridian pathway of the eyes, ears, or nose is improving. Thus, the status of the eyes, ears, or nose is determined based on changes in skin temperature at the Meridian pathway approximate to the eyes, ears, or nose.

### CROSS-REFERENCE TO RELATED APPLICATIONS

This non-provisional application claims priority under 35 U.S.C. § 119(a) on US Provisional Patent Application No(s). 62/007,554 filed on Apr. 9, 2020, the entire contents of which are hereby incorporated by reference.

### FIELD OF THE INVENTION

The present invention relates generally to a method for solid water particle treatment of eye, ear, and nose ailments. More so, a method for detecting and treating ailments in the eyes, ears, and nose utilizes an image capturing device to capture an initial infrared image of at least one hot spot near a Meridian pathway to visually indicate a skin temperature approximate to the affected meridian pathway at the eyes, ears, or nose; and then administer a predetermined dosage of solid water particle solution, whereby the solid water particle solution helps repair the Meridian pathway at the eyes, ears, or nose; and further capture a subsequent infrared image at or near the eyes, ears, or nose; and then compare the initial infrared image with the subsequent infrared image to determine the variance in temperature distribution at the eyes, ears, or nose; and finally determine the presence of ailments in the eyes, ears, or nose based on the variance in temperatures.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In general, the human body has Meridian vessels with clearly defined pathways that penetrate deep into the body. The Meridian vessels are the branches of the meridian pathway.

Typically, there are twelve major meridians that run on each side of the body, with one side mirroring the other. Each meridian corresponds to an internal organ; and each organ has its own physiological and invisible energy functions. The meridians are not only dependent on the other organs, but also on the greater meridian network. Because of the central and integral location of the meridian vessels/pathway, and because of the connectivity to the major organs, it is possible to determine the nascent stages of cancer.

Typically, Chakras are spiritual energy centers located in the spiritual bodies that channel Qi/Ki (In and Out) of the body's spiritual self. There are seven Chakras located at various points along the spine. The Chakras correlate with the Meridian pathway along the spine. Chakras are considered both transmitters and receivers of energy. The meridian pathway serves as an energy pathway in the body in which Qi/Ki flows through. It is known in the art that there must exist balance between Chakras and Meridians in order to be physically, emotionally, mentally, and spiritually healthy. Monitoring the Chakra in conjunction with the Meridian is also beneficial for detecting cancer.

Generally, the temperature of the human body is determined by the temperature of blood, which emits heat as far-infrared radiation. It is recognized that fat tissue absorbs farinfrared and the body is virtually completely protected with a layer of fat tissue adherent to the skin. Thus, measurement of temperature using the skin can achieve sufficient accuracy because sensors placed on skin characterized by the presence of fat tissue. A thermal imaging device can easily record and display the various colors associated with thermal energy levels on the surface of the skin.

Other proposals have involved nontraditional methods for treating eye, ear, and nose ailments. The problem with these treatment methods is that they do not detect and treat the ailments along the Meridian pathway. Also, solid water particles are not used to treat the ailments. Even though the above cited eye, ear, and nose treatment methods meet some of the needs of the market, a method for detection and treatment of ailments to the eyes, ears, and nose along the Meridian pathway that utilizes an image capturing device for capturing infrared images of hot spots near the Meridian pathway in the head region, so as to indicate irregularities in skin temperature; and then administering a predetermined amount of solid water particle solution approximate to the affected meridian pathway; and then comparatively capturing subsequent infrared images to determine presence of ailments and efficacy of treatment thereof, is still desired.

### SUMMARY

Illustrative embodiments of the disclosure are generally directed to a method for solid water particle treatment of eye, ear, and nose ailments that works to detect and treat ailments in the eyes, ears, and nose through a novel procedure of capturing thermal images of the Meridian pathway near the eyes, ears, or nose, and then administering a solid water particle solution to help repair the Meridian pathway. The efficacy of the solid water particle solution is determined by capturing an initial image, and a subsequent image of the Meridian pathway near the eyes, ears, or nose. The skin temperature of both the initial and subsequent thermal images is visually represented as a thermal coloring pattern, such as a white-hot spot. If the temperatures detected from the initial to the subsequent thermal images decrease, the Meridian pathway of the eyes, ears, or nose is improving. In this manner, the status of the eyes, ears, and nose is determined based on changes in skin temperature at the Meridian pathway approximate to the eyes, ears, and nose.

In some embodiments, the method comprises an initial Step of capturing, with an image capturing device, an initial infrared image of a hot spot along a Meridian pathway approximate to the eyes, ears, or nose, so as to visually indicate a skin temperature approximate to the Meridian pathway at the eyes, ears, or nose. The image capturing device may include without limitation, a thermograph, an infrared imaging apparatus, and a heat detection apparatus.

The Meridian pathway is based on a meridian theory of traditional Chinese medicine for identifying at least one acupoint along the meridian pathway. The higher body surface temperature indicated by the hot spot on the meridian pathway is thus, a reflection of the temperature of internal organs and cells where the meridian pathways pass through.

The image capturing device detects and records the hot spots and hot lines on the body surface, which may be an indication of potential ailments in the eyes, ears, and nose. In one embodiment in which a thermograph is used to capture the thermal image, the hot spot is indicated by a white spot, and cooler areas showing normal cells are indicated by a green region. For example, if a thermograph shows large white spots under the eyes, this can be an indication of short-sightedness or far-sightedness.

Another Step may include determining if the skin temperature near the Meridian pathway of the eyes, ears, or nose falls outside a temperature distribution range, whereby the temperature deviance from the temperature distribution range indicates an ailment to the eyes, ears, or nose.

The method may then include a Step of administering a predetermined dosage of solid water particle solution if the skin temperature is outside the temperature distribution range, whereby the solid water particle solution helps repair the Meridian pathway at the eyes, ears, or nose. Then after a duration, the method includes a Step of capturing a subsequent infrared image of the Meridian pathway approximate to the eyes, the ears, or the nose, the subsequent infrared image visually indicating the temperature of the skin at the affected Meridian pathway of the eyes, ears, or nose. In alternative embodiments, a third infrared image is captured after a longer duration for further analysis of the Meridian pathway.

Another Step comprises comparing the skin temperature from the initial infrared image with the skin temperature from the subsequent infrared image. After this comparison of skin temperatures, a determination of the status of the eyes, ears, or nose based on the shape, size, and position of the hot spot is studied to determine the status of the potential ailment. A final Step comprises determining if the skin temperature from the subsequent infrared image decreases from the skin temperature of the initial infrared image, whereby a temperature decrease indicates efficacy of the solid water particle solution to repair the Meridian pathway.

One objective of the present invention is to detect ailments in the eyes, ears, or nose by observing the skin temperature along the Meridian pathway at or near the eyes, ears, or nose.

Another objective is to utilize a thermograph to identify abnormal skin temperatures.

Another objective is to administer solid water particles towards a specific region of the body, and specifically at the Meridian pathway.

Another objective is to administer solid water particles by a variety of routes of administration including: oral, intravenous, intramuscular, subcutaneous, intrathecal, topical, inhalation.

Yet another objective is to provide multiple delivery means for administering the solid water particles.

Yet another objective is to observe variances in skin temperature around the eyes, ears, or nose to determine if an ailment is present.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart diagram of an exemplary method for solid water particle treatment of eye, ear, and nose ailments, in accordance with an embodiment of the present invention;

FIGS. 2A and 2B illustrate thermograph images of a subject with a first type of eye ailments, where FIG. 2A shows the hot spot before treatment with a solid water particle solution, and FIG. 2B shows the eyes with an absence of the hot spot, in accordance with an embodiment of the present invention;

FIGS. 3A and 3B illustrate thermograph images of a subject with a second type of eye ailments, where FIG. 3A shows the hot spot before treatment with a solid water particle solution, and FIG. 3B shows the eyes with an absence of the hot spot, in accordance with an embodiment of the present invention;

FIGS. 4A and 4B illustrate thermograph images of a subject with a first type of right ear ailments, where FIG. 4A shows the hot spot before treatment with a solid water particle solution, and FIG. 4B shows the eyes with an absence of the hot spot, in accordance with an embodiment of the present invention;

FIGS. 5A and 5B illustrate thermograph images of a subject with left ear ailments, where FIG. 5A shows the hot spot before treatment with a solid water particle solution, and FIG. 5B shows the left ear with an absence of the hot spot, in accordance with an embodiment of the present invention;

FIGS. 6A and 6B illustrate thermograph images of a subject with a second type of right ear ailments, where FIG. 6A shows the hot spot before treatment with a solid water particle solution, and FIG. 6B shows the right ear with an absence of the hot spot, in accordance with an embodiment of the present invention;

FIGS. 7A and 7B illustrate thermograph images of a subject with a first type of nose ailments, where FIG. 7A shows the hot spot on the nose before treatment with a solid water particle solution, and FIG. 7B shows the nose with an absence of the hot spot, in accordance with an embodiment of the present invention; and

FIGS. 8A and 8B illustrate thermograph images of a subject with a second type of nose ailments, where FIG. 8A shows the hot spot on the nose before treatment with a solid water particle solution, and FIG. 8B shows the nose with an absence of the hot spot, as a result of solid water particle solution increasing blood flow to the Meridian pathway, in accordance with an embodiment of the present invention.

Like reference numerals refer to like parts throughout the various views of the drawings.

### DETAILED DESCRIPTION OF THE INVENTION

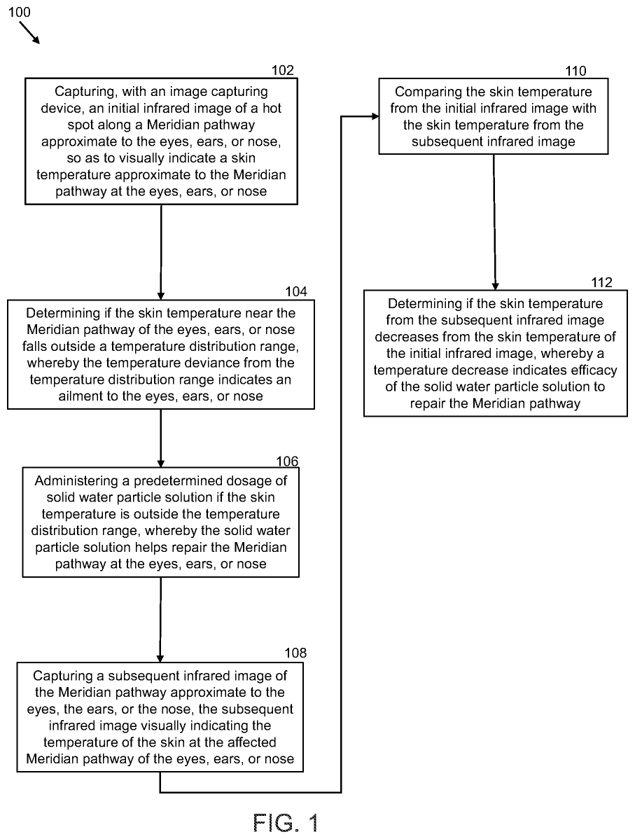

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A method 100 for solid water particle treatment of eye, ear, and nose ailments is referenced in FIGS. 1-8B. The method 100 for solid water particle treatment of eye, ear, and nose ailments, hereafter “method 100” is based on determining and repairing the Meridian pathway (network) of the body to heal a variety of eye, ear, and nose ailments. The method 100 includes the administration of a solid water particle solution to enhance performance of organs—and especially the eyes, ears, or nose—in proximation of the Meridian pathway. The method utilizes a thermal image 204 of the skin, showing heat and white spots, at and around the eyes, ears, or nose. The thermal images 204 of the hot spots are captured and compared to determine temperature deviations (potential ailments), and to determine the efficacy of the solid water particle solution.

Those skilled in the art will recognize that the Meridian pathway circulated throughout the body, including the eye, ear, and nose. When the Meridian pathway works properly, blood circulates fully to all the cells of eyes, ears, and nose. Thus, all the cells of eyes, ears, and nose receive sufficient nutrients from the blood to perform at optimal conditions and with no ailments. However, when the Meridian pathway does not have sufficient solid water particles, the blood does not circulate fully to all the cells of the eyes, ears, and nose.

Consequently, various ailments can degrade the eyes, ears and nose.

FIG. 1 references an exemplary flowchart of the method 100 for solid water particle treatment of eye, ear, and nose ailments. The method 100 includes a series of unique steps configured to detect and treat ailments in the eyes, ears, and nose through a procedure of capturing infrared images of the Meridian pathway near the eyes, ears, or nose, and then administering a solid water particle solution to help repair the Meridian pathway.

The efficacy of the solid water particle solution is determined by capturing an initial image and a subsequent image of the Meridian pathway near the eyes, ears, or nose. The skin temperature of both images is visually indicated in a thermal coloring or white-hot spot. If the temperatures detected in the infrared images decrease, the Meridian pathway of the eyes, ears, or nose is improving. Thus, the status of the eyes, ears, or nose is determined based on changes in skin temperature at the Meridian pathway approximate to the eyes, ears, or nose.

Looking again at flowchart in FIG. 1, the method 100 comprises an initial Step 102 of capturing, with an image capturing device, an initial infrared image of a hot spot along a Meridian pathway approximate to the eyes, ears, or nose, so as to visually indicate a skin temperature approximate to the Meridian pathway at the eyes, ears, or nose. In some embodiments, the hot spot may indicate ailments in the region of the head, such as the eyes, ears, or nose. The meridian pathway acts as a channel to the interior of body, where cancerous cells may be active. A white spot indicates an irregularly hot cell, indicating potential cancer.

It is known in the art that Meridian vessels are the branches of the meridian pathway that run on each side of the body, with one side mirroring the other. Each meridian corresponds to an internal organ, which can be an indicator of potential cancerous cells. The Meridian pathway (vessels) is based on a meridian theory of traditional Chinese medicine for identifying at least one acupoint along the meridian pathway. The higher body surface temperature indicated by the hot spot on the meridian pathway is thus, a reflection of the temperature of internal organs and cells where the meridian pathways pass through.

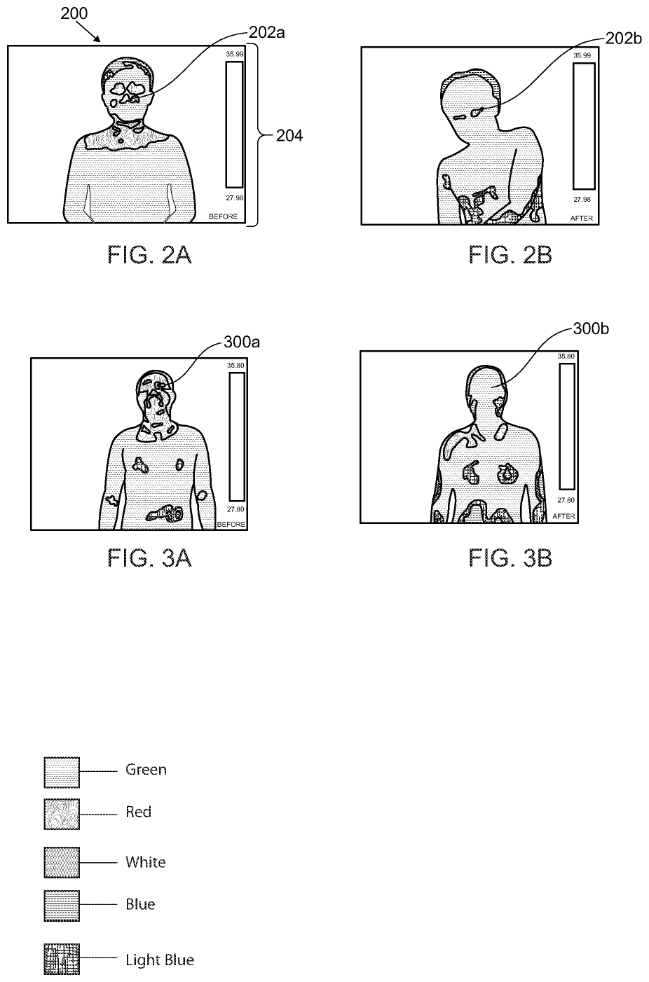

Specifically, the hot spots along the Meridian pathway are an indication of the excessive heat being generated by the eyes, ears, or nose. The image may be captured with an image capturing device, displaying various colors to show the hot spot and other thermal energy regions of the body. FIGS. 2A and 2B, for example, show ailments in the eyes that are captured in images of hot spots at or near Meridian pathways near the eyes, both hot spots before 202a, and hotspots after 202b treatment.

Those skilled in the art will recognize that eyes, ears, or nose emit hot infrared radiation, which are transmitted preferentially along the Meridian pathways and peaks at known acupoints that are close to the surface of the body. Thus, the method 100 measures the degree of increase in temperature of the eyes, ears, or nose by the quantitative temperature at the Meridian pathways. In essence, internal organ abnormalities can be revealed by taking thermographs of the body and corresponding problematic organs with the meridian pathways.

The image capturing device detects and records the hot spots and hot lines on the body surface, in the form of a thermal image 204. The configuration of the thermal image on the head region is an indication of potential ailments in the eyes, ears, and nose. For example, FIGS. 2A and 2B illustrate thermograph images of a subject with a first type of ailment in the eyes 200, such as far-sightedness or near-sightedness. FIG. 2A shows the hot spot at the eyes 202a before treatment with a solid water particle solution. The high level of heat indicates the first type of eye ailment. Then, after treatment with the solid water particle solution, FIG. 2B shows the eyes 202b with an absence, or at least substantial reduction, of the hot spots visible in FIG. 2A.

In some embodiments, the image capturing device may include without limitation, a thermograph, an infrared imaging apparatus, and a heat detection apparatus. In one embodiment the thermograph captures a thermal image. The hot spot is indicated by a white spot, where cooler areas showing normal cells are indicated by a green region. For example, if a thermograph shows large white spots under the eyes, this can be an indication of short-sightedness or farsightedness.

Those skilled in the art will recognize that infrared imaging is non-invasive, and is a passive image device. Infrared imaging relies on the infrared emitted by the body. Thus, multiple images of the body, and especially near the meridian pathways may be captured without any harm to the subject. The images are then immediately available for viewing. Some advantageous of using an image capturing device include, without limitation, the abnormal high temperature can be identified with simple training; the cost of the equipment is low; a sensitive imaging; and the easy viewing of early developing stage of cancers that other systems may not be able to detect.

Those skilled in the art will recognize that a thermograph is an instrument that produces a trace or image representing a record of the varying temperature or infrared radiation over an area or during a period of time. In one embodiment, the color code for the thermographs includes: white the hottest, then red, yellow, green, blue, and black as the coldest. A green color on the thermograph is adjusted to be the color for normal skin temperature.

For purposes of the present invention, abnormal high temperature is shown in white color, or the highest temperature of the body, which is about 2° Celsius lower or higher than normal skin temperature. In one embodiment, if the excessive high temperature which is in white color, also occurs in another area of well-known inflammation regions of the nose or ears, then there exists a potential for ailments associated with the eyes, ears, or nose.

Another Step 104 of the method 100 may include determining if the skin temperature near the Meridian pathway of the eyes, ears, or nose falls outside a temperature distribution range, whereby the temperature deviance from the temperature distribution range indicates an ailment to the eyes, ears, or nose. In some embodiments, the range outside the temperature distribution range comprises at least 2° Celsius above or below a skin temperature. The skin temperature is defined as being approximately between 33° Celsius and 37° Celsius. However, it is known in the art that the head region is cooler than the torso of the body; and thereby the skin temperature may be less near the eyes, ears, or nose for purposes of applying the method 100.

The method 100 may then include a Step 106 of administering a predetermined dosage of solid water particle solution if the skin temperature is outside the temperature distribution range, whereby the solid water particle solution helps repair the Meridian pathway at the eyes, ears, or nose. In one non-limiting embodiment, the solid water particle solution is administered towards a specific region of the body, and specifically at the Meridian pathway through use of a dropper or a sprayer. For example, a cylindrical-shaped sprayer having a volume of less than one-hundred ml serves to spray the solid water particle solution on the ears. However, in alternative embodiments, the solid water particle solution is administered through eclectic routes, including: oral, intravenous, intramuscular, subcutaneous, intrathecal, topical, inhalation.

Thus, the mechanism of healing by solid water particle solution may be explained simply as follows: Meridian pathways are hypothesized to consist of solid water particles (or stable water clusters). The malfunction of organs along meridians is explained in Chinese medicine as the blocking of the flow of Qi along the Meridian. By drinking solid water particles, the charged water repairs the meridian and unblocks it, so that qi can freely flow again.

In one embodiment, the solid water particle consists of only pure water molecules and no other chemicals, and generally does not have harmful side effects. The solid water particle may include, without limitation, stable water clusters, and IE. Those skilled in the art will recognize that solid water particles may be referred to in different scientific names, such as stable water clusters and IE crystals.

The delivery of the solid water particles may be performed through at least one of the following: oral administration, topical administration, vapor administration, and intravenous administration. In some embodiments, the administration of the solid water particles may take various forms, including, without limitation: drinking solution that contains solid water particles; rubbing cream that contains solid water particles; breathing in mist that contains solid water particles; spraying fine drops that contain solid water waters on skin; administering drops using eye droppers that contain solid water particles into affected area; and intravenous injection of solutions that contain solid water particles. The daily dosage depends on the severity of the cancer. The more severe, the state of the cancerous cell, the more dosage is required.

In some embodiments, the oral administration of the solid water particles comprises a liquid, a pill, or a gel consisting of the predetermined quantity of solid water particles. In other embodiments, the topical administration comprises a cream consisting of the predetermined quantity of solid water particles. In yet other embodiments, the vapor administration comprises a nebulizer, a sprayer, or an inhaler consisting of the predetermined quantity of solid water particles. In other exemplary embodiments, the intravenous administration comprises an intravenous injection consisting of the predetermined quantity of solid water particles. And yet other embodiments of administering the solid water particles, cream may be applied to the eyes, ears, or nose.

Then after a duration, the method 100 includes a Step 108 of capturing a subsequent infrared image of the Meridian pathway approximate to the eyes, the ears, or the nose, the subsequent infrared image visually indicating the temperature of the skin at the affected Meridian pathway of the eyes, ears, or nose. In some embodiments, the duration may be 15 minutes. In alternative embodiments, a third infrared image is captured after a longer duration for further analysis of the Meridian pathway.

Thus, a subsequent hot spot is detected by utilizing the image capturing device, such as a thermograph, to capture a thermograph image on a portion of a body. The thermograph image may display at least one hot spot on the body. The hot spot may be indicative of an abnormality in skin temperature, which indicates an ailment to the eyes, ears, or nose.

Another Step 110 comprises comparing the skin temperature from the initial infrared image with the skin temperature from the subsequent infrared image. After this comparison of skin temperatures, a determination of the status of the eyes, ears, or nose based on the shape, size, and position of the hot spot is studied to determine the status of the potential ailment. A final Step 112 comprises determining if the skin temperature from the subsequent infrared image decreases from the skin temperature of the initial infrared image, whereby a temperature decrease indicates efficacy of the solid water particle solution to repair the Meridian pathway. In this manner it is possible to detect ailments in the eyes, ears, or nose by observing the skin temperature along the Meridian pathway at or near the eyes, ears, or nose.

For example, FIGS. 2A-8B illustrate thermal images of the head region for a subject.

The Meridian pathways along at least one head region, and specifically for the eyes, the ears, and the nose are shown. FIGS. 2A and 2B illustrate thermograph images of a subject with a first type of ailment in the eyes 200, such as far-sightedness or near-sightedness. FIG. 2A shows the hot spot at the eyes 202a before treatment with a solid water particle solution. The high level of heat indicates the first type of eye ailment. Then, after treatment with the solid water particle solution, FIG. 2B shows the eyes 202b with an absence, or at least substantial reduction, of the hot spots visible in FIG. 2A.

Similarly, FIGS. 3A and 3B illustrate thermograph images of a subject with a second type of eye ailments, such a macular degeneration, cataracts, or glaucoma. For these types of eye ailments, FIG. 3A shows the hot spot on the eye 300a before treatment with a solid water particle solution. And FIG. 3B shows the eyes 300b with an absence of the hot spot after the solid water particle treatment.

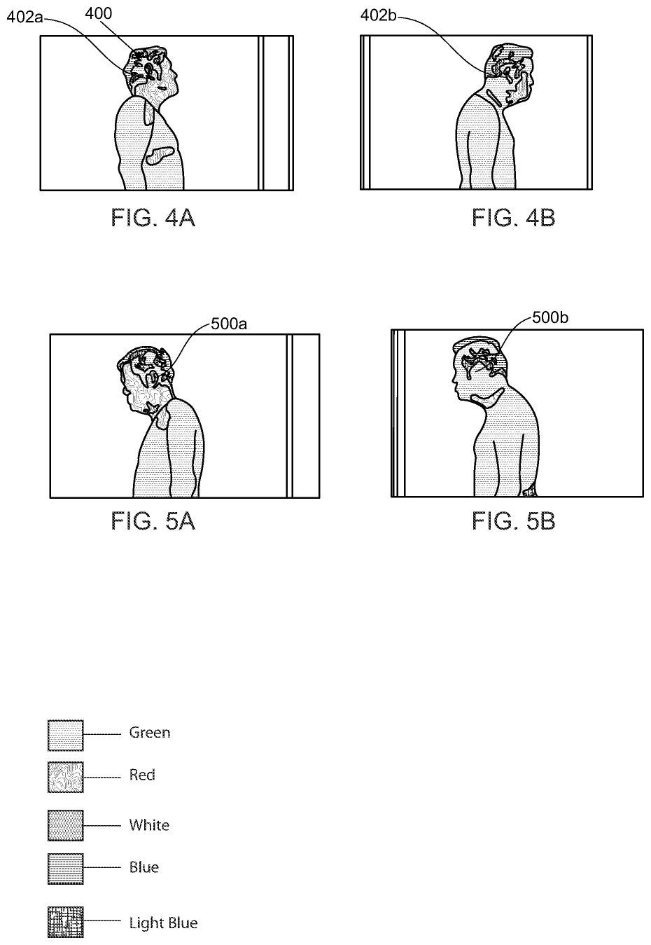

In addition to the eyes, the method 100 is operable to treat ailments to the ears 400 in substantially the same manner. For example, FIG. 4A references a thermograph images of a subject with an inflammation to the right ear. FIG. 4A shows the hot spot at the right ear 402a before treatment with a solid water particle solution, and FIG. 4B shows the eyes 402b with an absence of the hot spot. Looking now at the opposite ear, FIGS. 5A and 5B illustrate thermograph images of a subject with left ear ailments, where FIG. 5A shows the hot spot on the left ear 500a before treatment with a solid water particle solution, and FIG. 5B shows the left ear 500b with an absence of the hot spot.

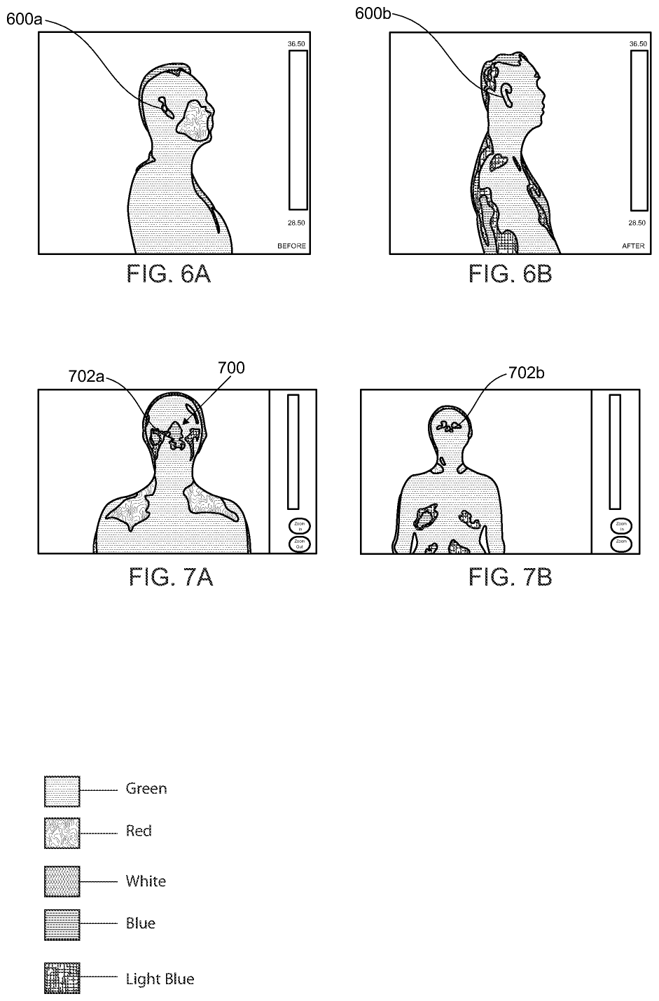

There are additional ailments to the ear that can be treated, beyond inflammations. For example, FIGS. 6A and 6B illustrate thermograph images of a subject with a second type of right ear ailments—loss of hearing, or loss of balance. FIG. 6A shows the hot spot of the right ear 600a before treatment with a solid water particle solution, and FIG. 6B shows the eyes 600b with an absence of the hot spot. However, in other embodiments, the method 100 is efficacious for treating additional types of ear ailments known in the art.

In addition to the eyes 200 and ears 400, the method 100 is operable to treat ailments to the nose 700 in substantially the same manner. FIGS. 7A and 7B illustrate thermograph images of a subject with a first type of ailment to the nose 700. This ailment may include an allergy. FIG. 7A shows the hot spot on the nose 702a before treatment with a solid water particle solution, and FIG. 7B shows the nose 702b with an absence of the hot spot.

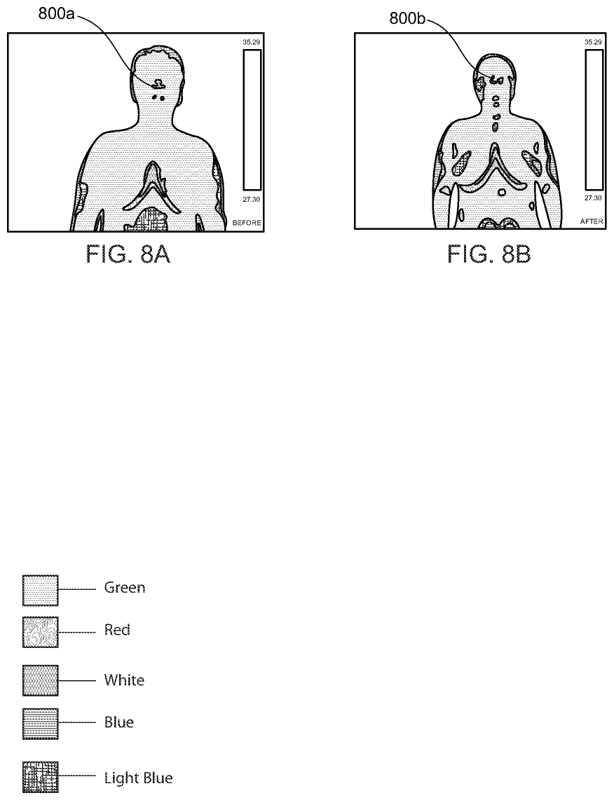

FIGS. 8A and 8B illustrate thermograph images of a subject with a second type of nose ailments. Then second type of nose ailment may include, without limitation, rhinitis, sinusitis, or a nasal poly. Here, FIG. 8A shows the hot spot on the nose 800a before treatment with a solid water particle solution, and FIG. 8B shows the nose 800b with an absence of the hot spot, as a result of solid water particle solution increasing blood flow to the Meridian pathway.

Thus, as the thermal images 204 in FIGS. 2A-8B show, the effectiveness of solid water clusters can be confirmed by the first and second infrared images (or thermographs). First, an initial body surface temperature infrared images are taken with no clothes on. Then, application of solid water particles, such as drinking a specific amount of water that contains large number of solid water particles. In one embodiment the large number could be more than three million solid water particles that are larger than 0.1 micron.

Thereafter, a subsequent infrared image of the body, after a duration, to determine a difference in temperature distribution on the skin. The duration may be fifteen minutes. The fifteen minutes is the minimal time required for the solid water particles to enter the body and diffuse through the body. Then a comparison between the first and second infrared images is made to determine the difference of temperature distribution on the skin, and especially the area in the eyes, ears, or nose, where the hot spots appear.

These and other advantages of the invention will be further understood and appreciated by those skilled in the art by reference to the following written specification, claims and appended drawings.

In some embodiments, this invention only demonstrates the method for solid water particle treatment of eye, ear, and nose ailments, the method comprising: administering a predetermined dosage of a solid water particle solution to eyes, ears, or nose, organs themselves, or the region around them, or acupoints near them.

In some embodiments, this invention only demonstrates the method for detecting possible inflamed eye, ear, and/or nose, the method comprising: First step: capturing, with an image capturing device, an initial infrared image of a hot spot along a Meridian pathway approximate to the eyes, ears, or nose, whereby the infrared image indicates a skin temperature.

Second step: determining if the skin temperature near the Meridian pathway of the eyes, ears, or nose falls outside a temperature distribution range, whereby the temperature deviance from the temperature distribution range indicates an ailment to the eyes, the ears, or the nose, whereby outside the temperature distribution range comprises at least 2 degrees Celsius above or below a skin temperature, the skin temperature is approximately between 33 degrees Celsius and 37 degrees Celsius.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method for solid water particle treatment of eye, ear, and nose ailments, the method comprising:
administering a predetermined dosage of a solid water particle solution to eyes, ears, or nose, organs themselves, or the region around them, or acupoints near them.

2. The method of claim 1, wherein an ailment to the eyes includes at least one of the following: far-sightedness, near-sightedness, cataract, glaucoma, and macular degeneration.

3. The method of claim 1, wherein an ailment to the ears comprises an inflammation.

4. The method of claim 1, wherein an ailment to the ears comprises a loss of body balance.

5. The method of claim 1, wherein an ailment to the ears comprises a loss in hearing.

6. The method of claim 1, wherein an ailment to the nose comprises an inflammation caused by at least one of the following: an allergy, rhinitis, sinusitis, and a nasal poly.

7. The method of claim 1, wherein the solid water particle solution is operable to help repair the Meridian pathway near the eyes, the ears, and the nose.

8. The method of claim 1, further comprising a step of administering the solid water particle solution with a dropper.

9. The method of claim 1, further comprising a step of administering the solid water particle solution with a sprayer.

10. The method of claim 9, wherein the sprayer comprises a cylindrical shape and a volume of less than 100 milliliters.

11. A method for solid water particle treatment of eye, ear, and nose ailments, the method comprising:
capturing, with an image capturing device, an initial infrared image of a hot spot along a Meridian pathway approximate to the eyes, ears, or nose, whereby the infrared image indicates a skin temperature;
determining if the skin temperature near the Meridian pathway of the eyes, ears, or nose falls outside a temperature distribution range, whereby the temperature deviance from the temperature distribution range indicates an ailment to the eyes, the ears, or the nose;
administering a predetermined dosage of a solid water particle solution if the skin temperature is outside the temperature distribution range;
capturing a subsequent infrared image of the Meridian pathway approximate to the eyes, the ears, or the nose, the subsequent infrared image visually indicating the temperature of the skin at the affected Meridian pathway of the eyes, ears, or nose;
comparing the skin temperature from the initial infrared image with the skin temperature from the subsequent infrared image; and
determining if the skin temperature from the subsequent infrared image decreases from the skin temperature of the initial infrared image.

12. The method of claim 11, wherein outside the temperature distribution range comprises at least 2 degrees Celsius above or below a skin temperature.

13. The method of claim 12, wherein the skin temperature is approximately between 33 degrees Celsius and 37 degrees Celsius.

14. A method for detecting possible inflamed eye, ear, and/or nose, the method comprising:
capturing, with an image capturing device, an initial infrared image of a hot spot along a Meridian pathway approximate to the eyes, ears, or nose, whereby the infrared image indicates a skin temperature;
determining if the skin temperature near the Meridian pathway of the eyes, ears, or nose falls outside a temperature distribution range, whereby the temperature deviance from the temperature distribution range indicates an ailment to the eyes, the ears, or the nose, whereby outside the temperature distribution range comprises at least 2 degrees Celsius above or below a skin temperature, the skin temperature is approximately between 33 degrees Celsius and 37 degrees Celsius;
